(19) United States
(12) Patent Application Publication
(10) Pub. No.: US 2026/0102342 A1
(43) Pub. Date: Apr. 16, 2026

(54) PILLING PASTE FORMULATION FOR THE ORAL ADMINISTRATION OF A MEDICATION TO AN ANIMAL

(71) Applicant: Tevra Brands, LLC, Omaha, NE (US)

(72) Inventor: Mark Levin, Omaha, NE (US)

(21) Appl. No.: 18/915,100

(22) Filed: Oct. 14, 2024

(51) Int. Cl. A61K 9/06; A61K 9/00; A61K 47/12; A61K 47/26; A61K 47/36; A61K 47/38; A61K 47/46

(52) U.S. Cl. CPC A61K 9/06; A61K 9/0053; A61K 47/12; A61K 47/26; A61K 47/36; A61K 47/38; A61K 47/46

(57) ABSTRACT

A pilling paste formulation and method of making the pilling paste formulation for the oral administration of a medication to an animal are disclosed. The pilling paste formulation comprises adhesive properties. The pilling paste formulation adheres to oral medication inserted into the adhesive pilling paste for administration of the medication to an animal.

### FIELD OF THE INVENTION

The present disclosure provides a pilling paste formulation for the oral administration of a medication.

### BACKGROUND OF THE INVENTION

In animal medicine, oral administration of a medication is often preferred over other routes. For instance, the oral route requires less specialized training than administration of a medication by the intravenous route, thus making it suitable for pet owners to administer a medication without veterinary assistance. However, oral administration of a medication is not without its challenges. When an animal rejects an oral medication, the animal may not receive the complete course of therapy, which results in poor outcomes for the treatment. Oral medications are often rejected by animals because of the taste, smell, and texture of the medication, all of which an animal may find unpalatable.

Earlier attempts at formulating pilling treats have been unable to successfully prevent detachment of the medication from the treat when ingested by the animal. Animals can easily detect the medication, separate the medication from treat components, and consume the treat but expel the medication. This is because earlier attempts at formulating treats for oral administration have been unable to successfully prevent detachment of the medication from the treat when ingested by the animal. Pilling treats can either be single textured or be multi-textured.

To date, single textured pilling treats are dough-like and do not stick to pills, tablets, capsules, or gel-caps. They are generally used without dispenser.

Existing approaches to improve the textural stability and palatability of pet pilling treats have had limited performance success for those animals that tend to chew their foods and treats before swallowing (often referred to as “Chewers”). “Chewers will bite down on all treats containing medications and quickly learn that they can remove the treat with their tongue and spit the medication out. The current pilling treats do not stick to the medications and are easily removed from the medication.

What is needed, therefore, is an animal treat composition with improved adhesive properties so that the animal treat adheres tightly to the oral medication. It is further desirable and an object of the invention to provide a pilling paste formulation that is able to adhere to the oral medication.

### BRIEF SUMMARY

One aspect of the present disclosure encompasses a pilling paste formulation. The pilling paste formulation comprises one or more sugars, one or more thickening agents, an acidifier, a preservative, one or more viscosity enhancing agents, or combinations thereof. The pilling paste formulation can be in direct contact to a pill, tablet, capsule, or soft gel.

Another aspect of the present disclosure encompasses a method of administering an oral medication to an animal. The method comprises inserting the oral medication into the pilling paste formulation. The animal may be a companion animal. Companion animals include but are not limited to dogs and cats.

Another aspect of the present disclosure encompasses a method of preparing a pilling paste formulation. The method comprises blending pre gelatinized dry ingredients to form a dry blend; blending pre gelatinized liquid ingredients to form a liquid blend; and blending the dry blend and the liquid blend to form the pilling paste formulation.

Another aspect of the present disclosure encompasses a method of administering a pilling paste formulation to an animal. The method comprises contacting an oral medication with the pilling paste formulation to form a coated oral medication, wherein at least a portion of the pilling paste formulation is in contact with at least one surface of the oral medication; and administering the coated oral medication to the animal.

Another aspect of the present disclosure encompasses a method of administering a pilling paste formulation through a dispenser, coating an oral medication, and presenting to an animal. The method comprises inserting a pilling paste formulation into a dispenser; ejecting the pilling paste formulation from the dispenser to form a dispensed pilling paste formulation; contacting the oral medication with the dispensed pilling paste formulation to form a coated oral medication, wherein at least a portion of the pilling paste is in contact with at least one surface of the oral medication; and administering the coated oral medication to the animal.

Another aspect of the present disclosure encompasses a pilling paste product. The pilling paste product comprises a pilling paste formulation coating an oral medication, wherein the pilling paste formulation comprises one or more sugars, one or more thickening agents, an acidifier, a preservative, one or more viscosity enhancing agents, or combinations thereof, and wherein the pilling paste formulation is in contact with at least one surface of the oral medication.

Another aspect of the present disclosure encompasses a pilling paste product. The pilling paste product comprises a pilling paste formulation that adheres to an oral medication upon contact. The pilling paste formulation comprises one or more sugars, one or more thickening agents, an acidifier, a preservative, one or more viscosity enhancing agents, or combinations thereof.

### DETAILED DESCRIPTION

The present invention relates to pilling paste formulations for administering an oral medication to an animal. The present invention also relates to a method of making the pilling paste formulations. The present invention also relates to a method of administering an oral medication using the disclosed pilling paste formulation.

### I. Pilling Paste Formulation

The pilling paste formulation is a sticky semi-solid mass characterized by a sufficient viscosity that is solid but malleable with minimal pressure. For instance, the pilling paste formulation can have the texture of a firm gel, a paste, or have an elastic texture. The pilling paste formulation is designed to receive an oral medication. The oral medication is inserted into the semi-solid mass with minimal force. The composition's adhesive portion is also characterized by adhesiveness that allows the adhesive portion to adhere to the oral medication and to prevent the animal from separating the oral medication from the animal treat composition.

The pilling paste formulation has an adhesive strength capable of retaining a medication when administered to an animal and prevent the animal from expelling the medication. The adhesive strength can be measured by, e.g., attaching a medication to a composition of an inner portion, and determining the amount of the composition adhered to the medication when the medication is removed from the inner portion. The adhesive strength of the pilling paste formulation can and will differ depending on the composition of the pilling paste formulation and the medication, among other variables.

In one aspect, a pilling paste formulation includes one or more sugars, one or more thickening agents, an acidifier, a preservative, one or more viscosity enhancing agents, or combinations thereof.

The pilling paste formulation may also include further includes a flavor. The flavor may be chicken flavor, beef flavor, peanut flavor, or a combination thereof.

The sugar provides sweetness to the pilling paste formulation, provides water binding and texture softening, and also adds to the stickiness of the pilling paste formulation, which is important for holding the oral medication to the pilling paste formulation of the treat. Sugars suitable for the pilling paste formulation of the animal treat composition can be dried or liquid starch hydrolysates such as: corn syrup, rice syrup, tapioca syrup, or molasses, honey, dextrose, glycerin, propylene glycol, maple sugar, maltose, sucrose, lactose, xylitol, glucose, or fructose used individually or in combination. Granulated white or brown sugar may also be used.

The one or more sugars may be glycerin, liquid or dried starch hydrolysate such as, corn syrup, rice syrup, tapioca syrup, liquid or dried molasses, honey, sugar, dextrose, propylene glycol, sorbitol, or combinations thereof. In some examples, the sugar may be glycerin. In other examples, the sugar may be rice syrup. In other embodiments, the one or more sugars may be a combination of glycerin and rice syrup.

The one or more sugars may range from about 5% to about 70% by weight (% w/w) of the total formulation. For example, the sugar may range from about 10% w/w to about 65% w/w, about 15% w/w to about 60% w/w, about 20% w/w to about 55% w/w, about 25% w/w to about 50% w/w, about 30% w/w to about 45% w/w, about 35% w/w to about 40% w/w, or about 37% w/w to about 39% w/w. In some embodiments, the amount of the sugar may be 15% w/w. In some embodiments, the amount of the sugar may be about 16% w/w.

In some examples, glycerin may range from about 10% to about 20% by weight of the total formulation. In some examples, the rice syrup may range from about 30% to about 40% by weight of the total formulation.

The one or more thickening agent may be polysaccharides, agar, carrageenan, cassia gum, carboxy methyl cellulose (CMC), gellan gum, guar gum, konjac gum, locust bean gum, methyl cellulose, hydroxy propyl methyl cellulose (HPMC), pectin, starch, glucomannan, galactomannan, xanthan gum, carrageenan, or a combination thereof. In some examples, the thickening agent may be guar gum. In other examples, the thickening agent may be locust bean gum. In yet other examples, the thickening agent may be a combination of guar gum and locust bean gum.

The one or more thickening agents may range from about 0.01% to about 5.0% by weight of the total formulation. For example, the thickening may range from about 0.05% w/w to about 4.5% w/w, about 0.1% w/w to about 4.0% w/w, about 1.05% w/w to about 3.5% w/w, about 1.1% w/w to about 3.0% w/w, about 1.15% w/w to about 2.5% w/w, about 1.2% w/w to about 2.0% w/w, or about 1.5% w/w to about 1.7% w/w. In some embodiments, the amount of one or more thickening agents may be about 0.8% w/w.

In some embodiments, the amount of guar gum may be about 0.15% w/w. In other embodiments, the amount of locust bean gum may be about 0.75% w/w. In yet other embodiments, the amount of guar gum may be about 0.15% w/w and the amount of locust bean gum may be about 0.75% w/w.

The acidifier may range from about 0.01% to about 5.0% by weight of the total formulation. For example, the acidifier may range from about 0.05% w/w to about 4.5% w/w, about 0.1% w/w to about 4.0% w/w, about 1.05% w/w to about 3.5% w/w, about 1.1% w/w to about 3.0% w/w, about 1.15% w/w to about 2.5% w/w, about 1.2% w/w to about 2.0% w/w, or about 1.5% w/w to about 1.7% w/w. In some embodiments, the amount of acidifier may be about 1.0% w/w.

The preservative may be cultured products. The cultured products may include organic acids. The preservative may be benzoic acid, sorbic acid, propionic acid, acetic acid, diethyl pyrocarbonate, sorbate salts, menadione, including the salts and esters thereof, cultured skim milk, cultured whey, cultured dextrose, cultured wheat flour, cultured rice flour, or other cultured products or a combination thereof. In some examples, the preservative is cultured skim milk.

The preservative may range from about 0.1% to about 5.0% by weight of the total formulation. For example, the preservative may range from about 0.05% w/w to about 4.5% w/w, about 0.1% w/w to about 4.0% w/w, about 1.05% w/w to about 3.5% w/w, about 1.1% w/w to about 3.0% w/w, about 1.15% w/w to about 2.5% w/w, about 1.2% w/w to about 2.0% w/w, or about 1.5% w/w to about 1.7% w/w. In some embodiments, the amount of preservative may be about 1.75% w/w.

The one or more viscosity enhancing agents may be garbanzo bean flour, arrowroot flour, corn flour, corn starch, wheat flour, rice flour, katakuri starch, potato starch, sago, peanut flour, tapioca flour, or combinations thereof. In some examples, the viscosity enhancing agent may be rice flour.

In some aspects, the pilling paste formulation may be acidified. For instance, the pilling paste formulation may have pH ranging from about 3.9 to 5.5, preferably a pH ranging from about 4.0 to about 4.7. In some embodiments, the pH may be less than 4.8. In some examples the pH may be acidified to about 4.5. In other examples, the pH may be acidified to less than 4.25.

The pilling paste formulation may have a viscosity of about 20, 000 cps to about 4,000, 000 cps. For example, the viscosity may range from about 25,000 cps to about 3,500,000 cps, about 30,000 cps to about 3,000,000 cps, about 35,000 cps to about 2,500,000 cps, about 40,000 cps to about 2,000,000 cps, about 45,000 cps to about 1,500,000 cps, about 50,000 cps to about 1,000,000 cps, about 100,000 cps to about 900,000 cps, about 200,000 cps to about 800,000 cps, about 300,000 cps to about 700,000 cps, about 400,000 cps to about 600,000 cps, or about 500,000 cps to about 550,000 cps.

The pilling paste formulation comprises a fat content of about 5% w/w or lower, 3% w/w or lower, 2% w/w or lower, 1% w/w or lower, or about 0.5% w/w or lower. While not wishing to be bound by theory, a low-fat content as described herein contributes to the stickiness of the adhesive portion and the adhesive nature that the treat is capable of achieving. Further, a low-fat content reduces the need for an emulsifier in the adhesive portion.

The pilling paste formulation may have a fat content of about 1.00% w/w to about 5.00% w/w. For example, the fat content may range from about 1.05% w/w to about 4.95% w/w, about 1.10% w/w to about 4.90% w/w, about 1.15% w/w to about 4.85% w/w, about 1.20% w/w to about 4.80% w/w, about 1.25% w/w to about 4.75% w/w, about 1.30% w/w to about 4.70% w/w, about 1.35% w/w to about 4.65% w/w, about 1.40% w/w to about 4.60% w/w, about 1.45% w/w to about 4.55% w/w, about 1.50% w/w to about 4.50% w/w, about 1.55% w/w to about 4.45% w/w, about 1.60% w/w to about 4.40% w/w, about 1.65% w/w to about 4.35% w/w, about 1.70% w/w to about 4.30% w/w, about 1.75% w/w to about 4.25% w/w, about 1.80% w/w to about 4.20% w/w, about 1.85% w/w to about 4.15% w/w, about 1.90% w/w to about 4.10% w/w, about 1.95% w/w to about 4.05% w/w, 2.00% w/w to about 4.00% w/w, about 2.05% w/w to about 3.95% w/w, about 2.10% w/w to about 3.90% w/w, about 2.15% w/w to about 3.85% w/w, about 2.20% w/w to about 3.80% w/w, about 2.25% w/w to about 3.75% w/w, about 2.30% w/w to about 3.70% w/w, about 2.35% w/w to about 3.65% w/w, about 2.40% w/w to about 3.60% w/w, about 2.45% w/w to about 3.55% w/w, about 2.50% w/w to about 3.50% w/w, about 2.55% w/w to about 3.45% w/w, about 2.60% w/w to about 3.40% w/w, about 2.65% w/w to about 3.35% w/w, about 2.70% w/w to about 3.30% w/w, about 2.75% w/w to about 3.25% w/w, about 2.80% w/w to about 3.20% w/w, about 2.85% w/w to about 3.15% w/w, about 2.90% w/w to about 3.10% w/w, or about 2.95% w/w to about 3.05% w/w.

Water activity (A_{w}) is equal to the vapor pressure of a composition divided by the vapor pressure of pure water under the same conditions. Using this particular definition, pure distilled water has a water activity of exactly one, and the water activity of a food is below one and is dependent on the degree to which water is bound in a food product. The pilling paste formulation may have a water activity of less than about 0.80. The pilling paste formulation may have a water activity of less than about 0.85. The pilling paste formulation may have a water activity of greater than or equal to about 0.60. The water activity may be less than about 0.725, about less than 0.70, or about less than 0.675. The water activity may be any number between about 0.60 to about 0.75. The water activity may be any number between about 0.50 to about 0.80.

### II. Pilling Paste Product

The present disclosure is also directed to a pilling paste product. The pilling paste formulation may include one or more sugars, one or more thickening agents, an acidifier, a preservative, one or more viscosity enhancing agents, or combinations thereof. The pilling paste formulation may adhere to an oral medication upon contact. The pilling paste formulation may be in contact with at least one surface of the oral medication. In some embodiments, the pilling paste formulation may coat the oral medication. The oral medication may be a pill, tablet, capsule, or soft gel.

### III. Method of Using the Pilling Paste Formulation

In another aspect, the present disclosure encompasses a method for administering an oral medication to an animal using the pilling paste formulation described in Section I above.

In some embodiments, the method comprises inserting an oral medication into the pilling paste animal treat and administering the medicated treat to the animal. The animal can be a companion animal such as a dog or a cat. The oral medication can be any form of oral medication such as a tablet, capsule, soft gel, or pill.

In other embodiments, the method comprises contacting the oral medication with the pilling paste formulation to form a coated oral medication and administering the coated oral medication to the animal. At least a portion of the pilling paste formulation may be in contact with at least one surface of the oral medication.

In yet other embodiments, the method includes inserting a pilling paste formulation into a dispenser; ejecting the pilling paste formulation from the dispenser to form a dispensed pilling paste formulation; contacting the oral medication with the dispensed pilling paste formulation to form a coated oral medication; and administering the coated oral medication to the animal. At least a portion of the pilling paste may be in contact with at least one surface of the oral medication.

The method of providing the oral medication to an animal is beneficial because the animal treat composition adheres tightly to the oral medication such that once inserted into the adhesive portion, the adhesive portion adheres to the oral medication, and an animal cannot remove the oral medication from the animal treat composition.

### IV. Method of Making a Pilling Paste Formulation

The present disclosure is further directed to a method of preparing a pilling paste formulation. The method includes the following steps: blending dry ingredient to form a dry blend; blending liquid ingredients to form a liquid blend; optionally heating the liquid blend to a specified temperature to form a heated liquid blend; adding the dry blend to the liquid blend or heated liquid blend; and blending the dry blend and liquid blend or the heated liquid blend to form the pilling paste formulation.

The heating of the liquid blend may be optional. In some examples, it may be preferred to heat to 165 F or higher to achieve a thermal kill and to cause the starch to gelatinize. However, it may be possible to formulate with pregelatinized starches and use alternative gums (that do not require heating) to make this through a cold process. Notably with the cold process, pharma like quality programs and very tight controls on ingredients quality would be needed. The first heating step is preferred to include heating in the disclosed process to reduce the need for the costly ingredients, higher levels of sanitation, and more intense quality programs.

The liquid blend may be heated to a temperature of about 165° F. to about 210° F. For example, the liquid blend may be heated to about 166° F., about 167° F., about 168° F., about 169° F., about 170° F., about 171° F., about 172° F., about 173° F., about 174° F., about 175° F., about 176° F., about 177° F., about 178° F., about 179° F., about 180° F., about 181° F., about 182° F., about 183° F., about 184° F., about 185° F., about 186° F., about 187° F., about 188° F., about 189° F., about 190° F., about 191° F., about 192° F., about 193° F., about 194° F., about 195° F., about 196° F., about 197° F., about 198° F., about 199° F., about 200° F., about 201° F., about 202° F., about 203° F., about 204° F., about 205° F., about 206° F., about 207° F., about 208° F., about 209° F., or about 210° F.

The mixture of the dry blend and liquid blend may be heated to a temperature of between 140° F. to about 190° F. For example, the mixture may be heated to about 141° F., about 142° F., about 143° F., about 144° F., about 145° F., about 146° F., about 147° F., about 148° F., about 149° F., about 150° F., about 151° F., about 152° F., about 153° F., about 154° F., about 155° F., about 156° F., about 157° F., about 158° F., about 159° F., about 160° F., about 161° F., about 162° F., about 163° F., about 164° F., about 165° F., about 166° F., about 167° F., about 168° F., about 169° F., about 170° F., about 171° F., about 172° F., about 173° F., about 174° F., about 175° F., about 176° F., about 177° F., about 178° F., about 179° F., about 180° F., about 181° F., about 182° F., about 183° F., about 184° F., about 185° F., about 186° F., about 187° F., about 188° F., about 189° F., or about 190° F.

### V. Definitions

Unless otherwise defined, all technical and scientific terms used herein have the same meaning as is commonly understood by one of skill in the art to which this invention belongs at the time of filing. If specifically defined, then the definition provided herein takes precedent over any dictionary or extrinsic definition. Further, unless otherwise required by context, singular terms shall include pluralities, and plural terms shall include the singular. Herein, the use of “or” means “and/or” unless stated otherwise. All patents and publications referred to herein are incorporated by reference.

As used herein, the terms “about” and “approximately” designate that a value is within a statistically meaningful range. Such a range can be typically within 20%, more typically still within 25%, and even more typically within 50% of a given value or range. The allowable variation encompassed by the terms “about” and “approximately” depends on the particular system under study and can be readily appreciated by one of ordinary skill in the art.

As used herein, “a” and “an” mean one or more, unless otherwise indicated.

As used herein, “animals” refers to non-human vertebrates. Especially contemplated are companion animals. Examples of companion animals include, but are not limited to, cats, dogs, rabbits, ferrets, gerbils, hamsters, chinchillas, guinea pigs, and domesticated rats.

Although the invention described herein is susceptible to various modifications and alternative iterations, specific aspects thereof have been described in greater detail above. It should be understood, however, that the detailed description is not intended to limit the invention to the specific aspects disclosed. Rather, it should be understood that the invention is intended to cover all modifications, equivalents, and alternatives falling within the spirit and scope of the invention as defined by the claim language.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1: Method of Making Chicken Pilling Paste Formulation

A 150 g batch of chicken pilling paste formulation was prepared by dry blending all the dry ingredients. Next the liquid ingredients were blended and heated to 200° F. The blended dry ingredients were added to the heated liquid ingredients. The dry and liquid ingredients were blended until smooth at a hold temperature of between 160-170° F. for 3 to 5 minutes. The water activity (A_{w}) and viscosity were measured until target values were reached. The target for water activity is below 7.5. The target for hot viscosity during the process of making the pilling paste is 90,000-250,000 cps at 80° C. The viscosity will increase as it cools and through the first 30 days after making the pilling paste to a range of about 500,000-2,500,000 cps. The target pH is less than 4.5. Once the target values were reached, the formulation was sent to packaging.

The ingredients were heated to 195° F. to activate the gums.

Table 1 lists the ingredients and percent weight of each ingredient in the chicken pilling paste formulation.

TABLE 1
Ingredients and percent used in chicken pilling paste formulation.

| Ingredient | Weight percent (% w/w) |
|---|---|
| Emulsified Chicken + SD Chicken | 32.5 |
| (1:4 parts water reconstitution) | |
| Rice Syrup | 31 |
| Palatant #1 | 3 |
| Guar Gum | 0.15 |
| Locust Bean Gum | 0.75 |
| Acid Calcium Sulfate | 1.0 |
| Cultured Skim Milk | 1.75 |
| Glycerin | 15 |
| Rice Flour | 20.25 |
| Palatant #2 | 0.6 |

As seen in Table 1, water was not added as an independent ingredient to the chicken pilling paste formulation. However, water is present in the formulation because some of the ingredients have water. Emulsified chicken is the primary water source in the formulation. The emulsified chicken contains 68% w/w water. The dry ingredients contain about 12% w/w water. Rice syrup contains about 12% w/w water.

Spray dried (SD) chicken was reconstituted at a 4:1 ratio and mixed with the emulsified chicken.

### Example 2: Method of Making Beef Pilling Paste Formulation

A 150 g batch of beef pilling paste formulation was prepared by dry blending all the dry ingredients. Next the liquid ingredients were blended and heated to 200 F. The blended dry ingredients were added to the heated liquid ingredients. The dry and liquid ingredients were blended until smooth at a hold temperature of between 160-190° F. for 3 to 5 minutes. The water activity (aW) and viscosity were measured until target values were reached. The target for water activity is below 0.75. The target for hot viscosity is target pH is less than 4.25. Once the target values were reached, the formulation was sent to packaging.

The ingredients were heated to 195° F. to activate the gums.

Table 2 lists the ingredients and amount of each ingredient in the chicken pilling paste formulation.

TABLE 2
Ingredients and percent used in beef pilling paste formulation.

| | Weight percent |
|---|---|
| Ingredients | (%) |
| Emulsified Beef + SD Beef (1:2.8 | 35.6 |
| parts water reconstitution) | |
| Rice Syrup | 31 |
| Rice Flour | 19.35 |
| Glycerin | 16 |
| Palatant #3 | 3 |
| Cultured Skim Milk | 1.75 |
| Acid Calcium Sulfate | 1 |
| Locust Bean Gum | 0.75 |
| Guar Gum | 0.15 |

As seen in Table 2, water was not added as an independent ingredient to the beef pilling paste formulation. However, water is present in the formulation because some of the ingredients have water. Emulsified beef is the primary water source in the formulation. Emulsified beef contains 74% w/w water. The dry ingredients contain about 12% w/w water. Rice syrup contains about 12% w/w water.

Spray dried (SD) beef was reconstituted at a 2.8:1 ratio with the emulsified beef. The beef mixture was reconstituted at a 1:2.8 ratio with water.

### Example 3: Method of Making Peanut Pilling Paste Formulation

A 150 g batch of peanut pilling paste formulation was prepared by dry blending all the dry ingredients. Next the liquid ingredients except peanut flavor were blended and heated to 200° F. The blended dry ingredients were added to the heated liquid ingredients. The dry and liquid ingredients were blended until smooth at a hold temperature of between 160-190° F. for 3 to 5 minutes. The water activity (aW) and viscosity were measured until target values were reached. The target for water activity is below 0.75. The target for viscosity is 90,000-350,000 cps. The target pH is less than 4.25. Once the target values were reached, the formulation was sent to packaging.

The ingredients were heated to 195° F. to activate the gums.

Table 3 lists the ingredients and amount of each ingredient in the peanut pilling paste formulation.

TABLE 3
Ingredients and percent used in peanut pilling paste formulation.

| Ingredients | Weight (%) |
|---|---|
| Emulsified Chicken + SD Chicken | 35.5 |
| (1:4 parts water reconstitution) | |
| Rice Syrup | 31 |
| Rice Flour | 15.35 |
| Glycerin | 16 |
| Ground Peanuts | 3.5 |
| Cultured Skim Milk | 1.75 |
| Acid Calcium Sulfate | 1 |
| Natural Peanut Flavor | 1 |
| Locust Bean Gum | 0.75 |
| Guar Gum | 0.15 |

As seen in Table 3, water was not added as an independent ingredient to the peanut pilling paste formulation. However, water is present in the formulation because some of the ingredients have water. Emulsified Chicken is the primary water source in the formulation. Emulsified Chicken contains 74% w/w water. The dry ingredients contain about 12% w/w water. Rice syrup contains about 12% w/w water.

Spray dried chicken was reconstituted at a 4:1 ratio and added to the Emulsified chicken.

What is claimed is:

1. A pilling paste formulation comprising one or more sugars, one or more thickening agents, an acidifier, a preservative, one or more viscosity enhancing agents, or combinations thereof.

2. The pilling paste formulation of claim 1, wherein the pilling paste formulation is in direct contact to a pill, tablet, capsule, or soft gel.

3. The pilling paste formulation of claim 1, further comprising a flavor.

4. The pilling paste formulation of claim 1, wherein the one or more sugars ranges from about 10% to about 65% by weight of the total formulation.

5. The pilling paste formulation of claim 1, wherein the one or more thickening agents ranges from about 0.1% to about 2.0% by weight of the total formulation.

6. The pilling paste formulation of claim 1, wherein the acidifier ranges from about 0.5% to about 2% by weight of the total formulation.

7. The pilling paste formulation of claim 1, wherein the preservative from about 0.1% to about 2.0% by weight of the total formulation.

8. The pilling paste formulation of claim 1, wherein the one or more viscosity enhancing agents are selected from the group consisting of garbanzo bean flour, arrowroot flour, corn flour, corn starch, wheat flour, wheat starch, rice flour, rice starch, oat flour, soy, pea starch, pea protein, katakuri starch, potato starch, sago, peanut flour, tapioca starch, and tapioca flour.

9. The pilling paste formulation of claim 1, wherein the one or more sugars comprise glycerin, liquid or dried starch hydrolysate such as, corn syrup, rice syrup, tapioca syrup, liquid or dried molasses, honey, sugar, dextrose, propylene glycol, sorbitol, or combinations thereof.

10. The pilling paste formulation of claim 9, wherein the one or more sugars is rice syrup.

11. The pilling paste formulation of claim 9, wherein the one or more sugars is glycerin.

12. The pilling paste formulation of claim 9, wherein the one or more sugars in a combination of rice syrup and glycerin.

13. The pilling paste formulation of claim 11, the glycerin ranges from about 10% to about 20% by weight of the total formulation.

14. The pilling paste formulation of claim 10, wherein the rice syrup ranges from about 30% to about 40% by weight of the total formulation.

15. The pilling paste formulation of claim 1, wherein the one or more thickening agent comprise polysaccharides, agar, carrageenan, cassia gum, carboxy methyl cellulose (CMC), gellan gum, guar gum, konjac gum, locust bean gum, methyl cellulose, hydroxy propyl methyl cellulose (HPMC), pectin, starch, glucomannan, galactomannan, xanthan gum, carrageenan, or a combination thereof.

16. The pilling paste formulation of claim 15, wherein the thickening agent is guar gum.

17. The pilling paste formulation of claim 16, wherein the guar gum ranges from about 0.1% to about 2% by weight of the total formulation.

18. The pilling paste formulation of claim 15, wherein the thickening agent is locust bean gum.

19. The pilling paste formulation of claim 18, wherein the locust bean gum ranges from about 0.1% to about 2% by weight of the total formulation.

20. The pilling paste formulation of claim 1, wherein the preservative comprises cultured products.

21. The pilling paste formulation of claim 20, wherein the cultured products comprise organic acids.

22. The pilling paste formulation of claim 1, wherein the preservative is selected from the group consisting of acetic acid, benzoic acid, sorbic acid, propionic acid, diethyl pyrocarbonate, sorbate salts, menadione, including the salts and esters thereof, cultured whey, cultured wheat flour, cultured rice flour, cultured tapioca flour, cultured dextrose, and cultured skim milk.

23. The pilling paste formulation of claim 1, wherein the pilling paste formulation is acidified to a pH of about 5.0 or less.

24. The pilling paste formulation of claim 1, wherein the pilling paste formulation has a viscosity of about 50,000 cps to about 3,500,000 cps.

25. The pilling paste formulation of claim 24, wherein the pilling paste formulation has a viscosity of about 150,000 cps to about 2,500,000 cps.

26. The pilling paste formulation of claim 24, wherein the pilling paste formulation has a viscosity of about 200,000 cps to about 1,750,000 cps.

27. The pilling paste formulation of claim 24, wherein the pilling paste formulation has a viscosity of about 225,000 cps to about 1,500,000 cps.

28. The pilling paste formulation of claim 1, wherein the pilling paste has a fat content of about 1.0% w/w to about 4.75% w/w.

29. The pilling paste formulation of claim 1, wherein the pilling paste has a water activity ranging from about 0.55 to about 0.80.

30. A method of administering an oral medication to an animal the method comprising inserting the oral medication into the pilling paste formulation of claim 1.

31. The method of claim 30, wherein the animal is a companion animal.

32. The method of claim 30, wherein the animal is a dog.

33. The method of claim 30, wherein the animal is a cat.

34. A method of preparing a pilling paste formulation, the method comprising blending dry ingredient to form a dry blend;
blending liquid ingredients to form a liquid blend;
heating the liquid blend to a specified temperature to form a heated liquid blend;
adding the dry blend to the heated liquid blend; and
blending the dry blend and the heated liquid blend to form the pilling paste formulation.

35. A method of preparing a pilling paste formulation, the method comprising
blending pre gelatinized dry ingredients to form a dry blend;
blending pre gelatinized liquid ingredients to form a liquid blend; and
blending the dry blend and the liquid blend to form the pilling paste formulation.

36. A method of administering a pilling paste formulation to an animal, the method comprising
contacting an oral medication with the pilling paste formulation to form a coated oral medication, wherein at least a portion of the pilling paste formulation is in contact with at least one surface of the oral medication; and
administering the coated oral medication to the animal.

37. A method of administering a pilling paste formulation through a dispenser, coating an oral medication, and presenting to an animal, the method comprising:
inserting a pilling paste formulation into a dispenser;
ejecting the pilling paste formulation from the dispenser to form a dispensed pilling paste formulation;
contacting the oral medication with the dispensed pilling paste formulation to form a coated oral medication, wherein at least a portion of the pilling paste is in contact with at least one surface of the oral medication; and
administering the coated oral medication to the animal.

38. The method of any one of claim 36 or 37, wherein the oral medication is a pill, tablet, capsule, or soft gel.

39. A pilling paste product comprising a pilling paste formulation coating an oral medication, wherein the pilling paste formulation comprises one or more sugars, one or more thickening agents, an acidifier, a preservative, one or more viscosity enhancing agents, or combinations thereof, and wherein the pilling paste formulation is in contact with at least one surface of the oral medication.

40. The product of claim 39, wherein the oral medication is a pill, tablet, capsule, or soft gel.

41. A pilling paste product comprising a pilling paste formulation that adheres to an oral medication upon contact, wherein the pilling paste formulation comprises one or more sugars, one or more thickening agents, an acidifier, a preservative, one or more viscosity enhancing agents, or combinations thereof.

42. The product of claim 41 wherein the pilling paste formulation is in contact with at least one surface of the oral medication.

43. The product of claim 42, wherein the oral medication is a pill, tablet, capsule, or soft gel.
